(19) United States
(12) Patent Application Publication
(10) Pub. No.: US 2023/0132294 A1
(43) Pub. Date: Apr. 27, 2023

(54) ENDOSCOPE SYSTEM AND CONTROLLER

(71) Applicant: OLYMPUS CORPORATION, Tokyo (JP)

(72) Inventors: Hiroyuki FUKUDA, Machida-shi (JP); Jun TAKAHASHI, Hachioji-shi (JP); Reisuke OSADA, Hino-shi (JP); Chikayoshi MEGURO, Hachioji-shi (JP); Hiroyuki OTA, Yokohama-shi (JP); Tomoki HIRASAKA, Hino-shi (JP); Nanaho TOGI, Hachioji-shi (JP); Yui KAWANISHI, Koganei-shi (JP); Yukiko ASAI, Hachioji-shi (JP)

(73) Assignee: OLYMPUS CORPORATION, Tokyo (JP)

(21) Appl. No.: 17/969,128

(22) Filed: Oct. 19, 2022

Related U.S. Application Data
(60) Provisional application No. 63/272,338, filed on Oct. 27, 2021.

(51) Int. Cl. A61B 1/00; A61B 1/005; A61B 1/015

(52) U.S. Cl. CPC A61B 1/0016; A61B 1/0052; A61B 1/00042; A61B 1/015; A61B 1/00006; A61B 1/0004

(57) ABSTRACT

An endoscope system includes: an endoscope including a bending portion that is capable of bending; a drive device that is detachably connected to the endoscope and drives the endoscope; and an operation device that is communicably connected to the drive device and has an input portion that inputs an operation for driving the endoscope, wherein the drive device can select a method of controlling the bending portion with respect to the operation input from the input portion from fine movement control and coarse movement control, and the distance at which the drive device bends the bending portion by the coarse movement control with respect to a predetermined operation input is larger than the distance at which the drive device bends the bending portion by the fine movement control.

The present disclosure relates to an endoscope system. The present application claims priority based on U.S. Patent Provisional Application No. 63/272,338 provisionally filed in the United States on Oct. 27, 2021, the contents of which are incorporated herein by reference.

### BACKGROUND

### Technical Field

### Background Art

Conventionally, an endoscope has been used for observation and treatment in luminal organs such as digestive tract. There is a demand for an endoscope system that can perform observation and treatment using the endoscope more efficiently. In the manipulator system described in Japanese Patent No. 5192898, since a manipulator provided with the endoscope is electrically driven, observation and treatment by the endoscope can be efficiently performed.

In an endoscope system equipped with an electrically driven endoscope, it is desired that the operation portion (controller) into which operation by a surgeon is input is easy to handle and can be operated intuitively.

### SUMMARY

According to a first aspect of the present invention, an endoscope system includes: an endoscope including a bending portion that is capable of bending; a drive device that is detachably connected to the endoscope and drives the endoscope; and an operation device that is communicably connected to the drive device and has an input portion that inputs an operation for driving the endoscope, wherein the drive device can select a method of controlling the bending portion with respect to the operation input from the input portion from fine movement control and coarse movement control, and the distance at which the drive device bends the bending portion by the coarse movement control with respect to a predetermined operation input is larger than the distance at which the drive device bends the bending portion by the fine movement control.

According to a second aspect of the present invention, a controller controls an endoscope including a bending portion that is capable of bending, and includes a coarse movement input portion and a fine movement input portion, which are input portion that controls the bending portion, wherein the fine movement input portion and the coarse movement input portion are different types of input portion.

### BRIEF DESCRIPTION OF THE DRAWINGS

FIG. 1 is an overall view of an endoscope system according to a first embodiment.

FIG. 2 is a diagram showing an endoscope and an operation device of the endoscope system used by a surgeon.

FIG. 3 is a diagram showing an insertion portion of the endoscope.

FIG. 4 is a cross-sectional view of the bending portion.

FIG. 5 is a diagram showing the operation device.

FIG. 6 is a diagram showing the operation device as seen from the left side.

FIG. 7 is a diagram showing the operation device as seen from the right side.

FIG. 8 is a diagram showing the operation device as seen from the rear side.

FIG. 9 is a diagram showing the operation device held in a first position.

FIG. 10 is a diagram showing the operation device held in the first position.

FIG. 11 is a diagram showing the operation device held in the first position.

FIG. 12 is a diagram showing the operation device held in a second position.

FIG. 13 is a functional block diagram of a drive device of the endoscope system.

FIG. 14 is a functional block diagram of a video control device of the endoscope system.

FIG. 15 is a diagram showing an operation device of an endoscope system according to a second embodiment.

FIG. 16 is a diagram showing the operation device as seen from the left.

FIG. 17 is a diagram showing the operation device as seen from the right side.

FIG. 18 is a diagram showing the operation device as seen from the rear.

FIG. 19 is a diagram showing the operation device held by the left hand of the surgeon.

FIG. 20 is a diagram showing an operation device of an endoscope system according to a third embodiment.

FIG. 21 is a diagram showing the operation device as seen from the front.

FIG. 22 is a diagram showing a modified example of the operation device.

FIG. 23 is a diagram showing another modification of the operation device.

FIG. 24 is a diagram showing an operation device for an endoscope system according to a fourth embodiment.

FIG. 25 is a diagram showing an operation device of an endoscope system according to a fifth embodiment.

FIG. 26 is a diagram showing the operation device.

### DETAILED DESCRIPTION OF THE PREFERRED EMBODIMENTS

### First Embodiment

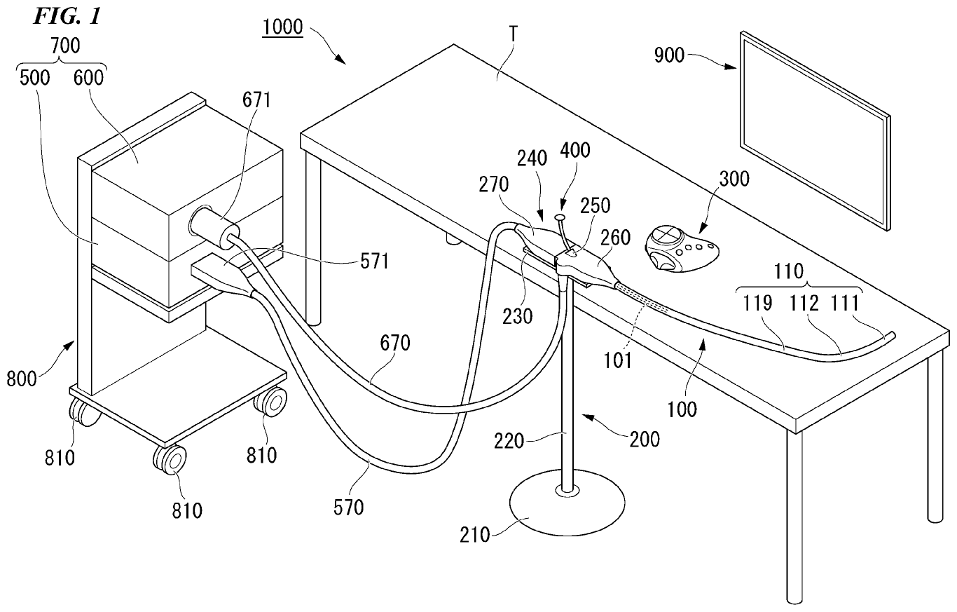

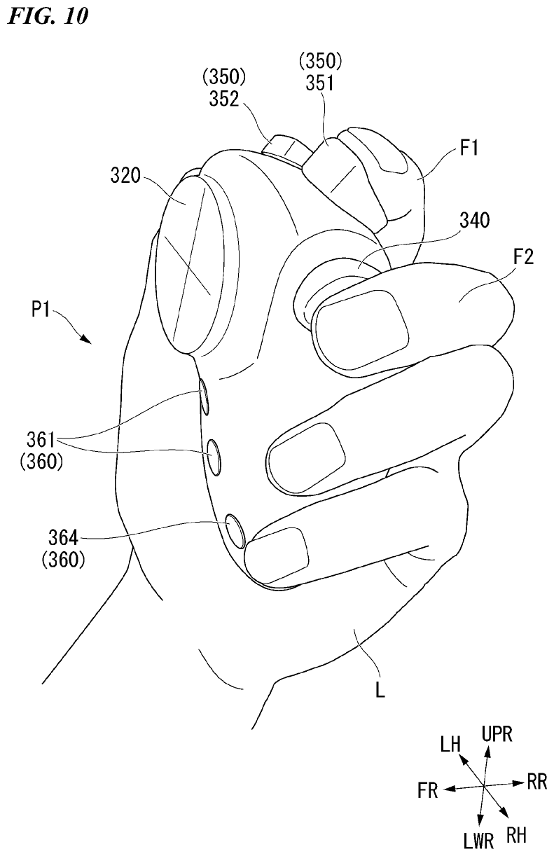

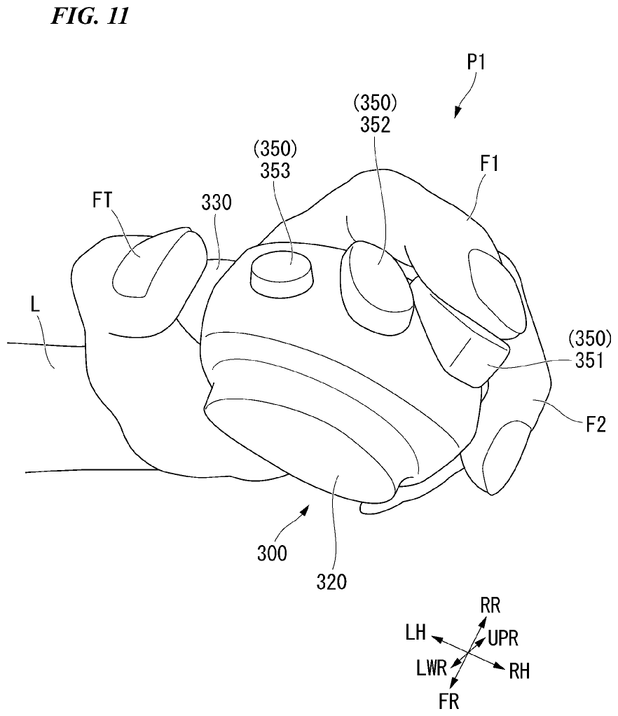

An endoscope system 1000 according to a first embodiment of the present invention will be described with reference to FIGS. 1 to 14. FIG. 1 is an overall view of the endoscope system 1000 according to the present embodiment.

### [Endoscope System 1000]

The endoscope system 1000 is a medical system that observes and treats the inside of the patient P lying on an operation table T. The endoscope system 1000 includes an endoscope 100, a stand 200, an operation device 300, a treatment tool 400, a drive device 500, a video control device 600, a trolley 800, and a display device 900. A drive cable 570 is connected to the drive device 500. A video control cable 670 is connected to the video control device 600.

The endoscope 100 is a device that is inserted into the lumen of patient P to observe and treat the affected area. The endoscope 100 is connected to the drive device 500 and the video control device 600 via the connector 240 included in the stand 200. An internal path 101 is formed inside the endoscope 100. In the following description, in the endoscope 100, the side inserted into the lumen of the patient P is referred to as “distal end side A1”, and the side connected to the connector 240 is referred to as “proximal end side A2”.

The stand 200 can be arranged at a position away from the trolley 800, and has a connector 240 for connecting the endoscope 100 to the drive cable 570 and the video control cable 670.

The operation device 300 is a device for inputting the operation of the surgeon who controls the endoscope system 1000. The operation device 300 can communicate with the drive device 500 and the like by wireless communication. The operation device 300 may be detachably connected to the drive device 500 via the operation cable 301 and may be able to communicate with the drive device 500. The surgeon S can electrically drive the endoscope 100 by operating the operation device 300.

The treatment tool 400 is a device that inserts the internal path 101 of the endoscope 100 into the lumen of the patient P to treat the affected area. In FIG. 1, the treatment tool 400 is inserted into the internal path 101 of the endoscope 100 via the forceps opening 250 formed in the connector 240.

The drive device 500 is detachably connected to the drive cable 570. The drive device 500 is connected to the endoscope 100 via the drive cable 570 and the connector 240. The drive device 500 drives the endoscope 100 by electrically driving the drive cable 570 using a built-in motor based on the operation input to the operation device 300. Further, the drive device 500 drives a built-in pump or the like based on the operation input to the operation device 300 to cause the endoscope 100 to perform air supply suction.

The video control device 600 is detachably connected to the video control cable 670. The video control device 600 is connected to the endoscope 100 via the video control cable 670 and the connector 240, and acquires a captured image from the endoscope 100. The video control device 600 causes the display device 900 to display the captured image acquired from the endoscope 100 and the GUI image or the CG image for the purpose of providing information to the surgeon.

The drive device 500 and the video control device 600 constitute a control device 700 that controls the endoscope system 1000. The control device 700 may further include peripheral devices such as a video printer. The drive device 500 and the video control device 600 may be an integrated device.

The trolley 800 is a trolley having wheels 810, and is equipped with a control device 700 (drive device 500 and video control device 600). The trolley 800 may be equipped with, for example, only the drive device 500. Further, the trolley 800 may have a hanger on which the operation device 300 and the like can be suspended.

The display device 900 is a device capable of displaying an image such as an LCD. The display device 900 is connected to the video control device 600 via a display cable.

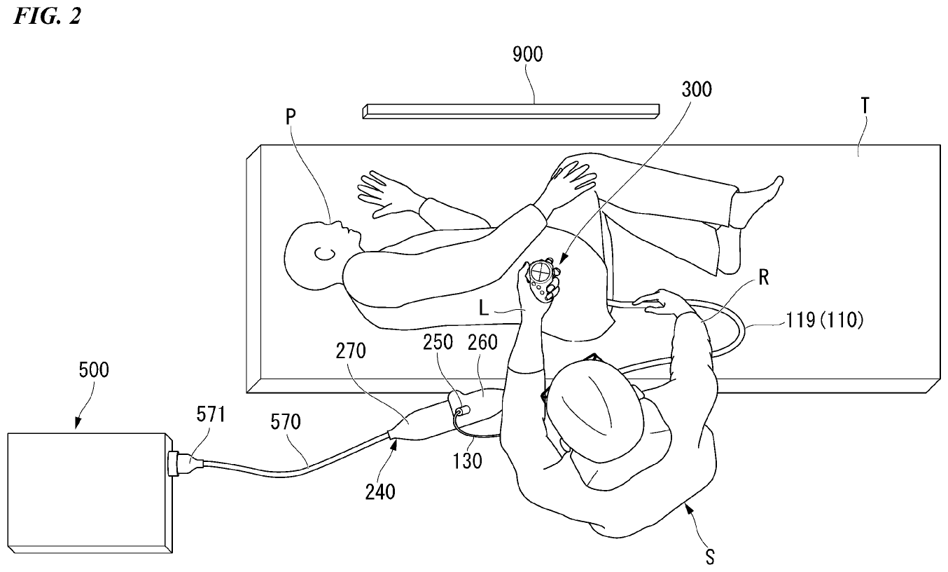

FIG. 2 is a diagram showing an endoscope 100 and an operation device 300 used by the surgeon S.

For example, the surgeon S operates the operation device 300 with the left hand L, for example, while observing the captured image displayed on the display device 900, while operating the endoscope 100 inserted into the lumen from the anus of patient P. Since the endoscope 100 and the operation device 300 are separated, the surgeon S can operate the endoscope 100 and the operation device 300 independently without being affected by each other.

Next, each device of the endoscope system 1000 will be described in detail.

### [Endoscope 100]

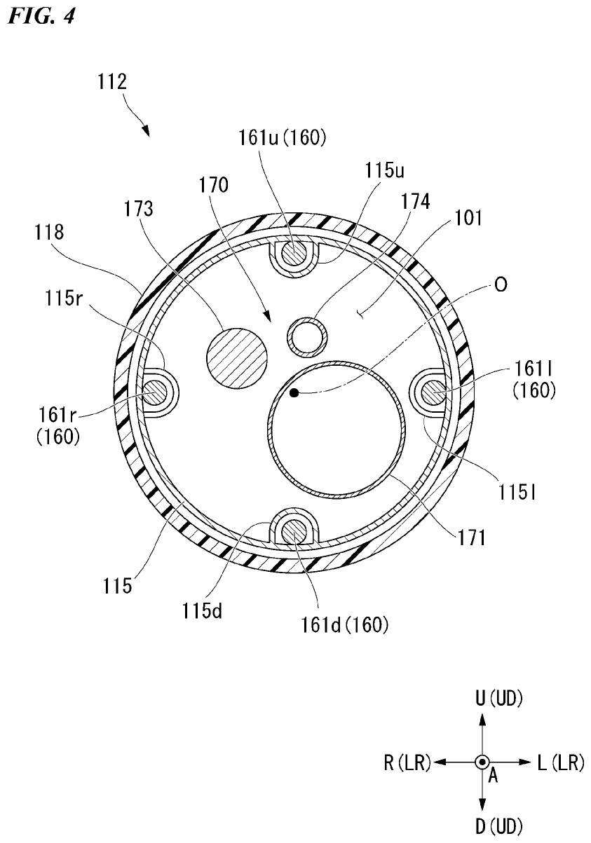

As shown in FIG. 1, the endoscope 100 includes an insertion portion 110, a curved wire 160 (see FIG. 4), and an internal component 170 (see FIG. 4). The proximal end of the endoscope 100 is connected to the connector 240.

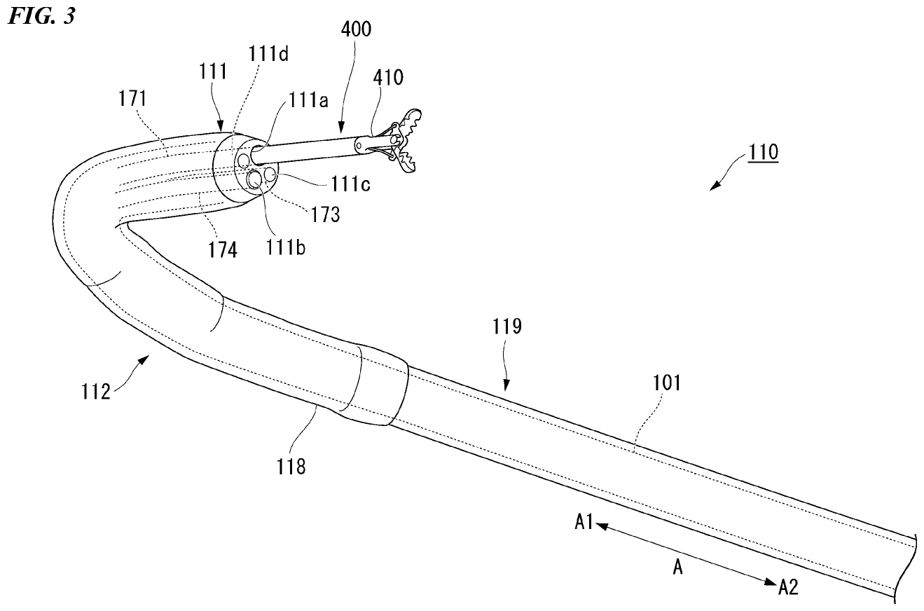

FIG. 3 is a diagram showing an insertion portion 110 of the endoscope 100.

Inside the endoscope 100, an internal path 101 extending from the distal end end of the insertion portion 110 to the proximal end along the longitudinal direction A of the endoscope 100 is formed. The curved wire 160 and the internal structure 170 are inserted into the internal path 101. The built-in object 170 includes a channel tube 171, an imaging cable 173, and a light guide 174.

The insertion portion 110 is an elongated long member that can be inserted into the lumen. The insertion portion 110 has a distal end portion 111, a bending portion 112, and an internal soft portion 119. The distal end portion 111, the bending portion 112, and the internal soft portion 119 are connected in order from the distal end side.

The distal end portion 111 is formed in a substantially cylindrical shape by metal or the like. As shown in FIG. 3, the distal end portion 111 has an opening portion 111a, an illumination portion 111b, and an imaging portion 111c. The opening 111a is an opening that communicates with the channel tube 171. As shown in FIG. 3, a treatment portion 410 such as a gripping forceps provided at the distal end of the treatment tool 400 through which the channel tube 171 is inserted is recessed from the opening 111a.

The illumination portion 111b is connected to a light guide 174 that guides the illumination light, and emits illumination light that illuminates the imaging target. The imaging portion 111c includes an imaging element such as CMOS, and takes an image of an imaging target. The imaging signal is sent to the video control device 600 via the imaging cable 173 and the video control cable 670.

FIG. 4 is a cross-sectional view of the bending portion 112.

The bending portion 112 has a plurality of knot rings (also referred to as curved pieces) 115. The plurality of knot rings 115 are connected so as to be bendable in the longitudinal direction A. The bending portion 112 can be curved in a vertical direction (also referred to as “UD direction”) perpendicular to the longitudinal direction A and in a left-right direction (also referred to as “LR direction”) perpendicular to the longitudinal direction A and the UD direction.

An upper wire guide 115u, a lower wire guide 115d, a left wire guide 1151, and a right wire guide 115r are formed on the inner peripheral surface of the knot ring 115. The upper wire guide 115u and the lower wire guide 115d are arranged on both sides in the UD direction with the central axis O in the longitudinal direction A interposed therebetween. The left wire guide 1151 and the right wire guide 115r are arranged on both sides in the LR direction with the central axis O in the longitudinal direction A interposed therebetween.

The curved wire 160 is a wire that bends the bending portion 112. The curved wire 160 extends through the internal path 101 to the connector 240. The curved wire 160 has an upper curved wire 161u, a lower curved wire 161d, a left curved wire 1611, and a right curved wire 161r.

The upper curved wire 161u and the lower curved wire 161d are wires that bend the bending portion 112 in the UD direction. The upper curved wire 161u is inserted with the upper wire guide 115u. The lower curved wire 161d is inserted with the lower wire guide 1l5d. The distal ends of the upper curved wire 161u and the lower curved wire 161d are fixed to the distal end portion 111.

The left curved wire 1611 and the right curved wire 161r are wires that bend the bending portion 112 in the LR direction. The left curved wire 1611 is inserted with the left wire guide 1151. The right curved wire 161r is inserted with the right wire guide 115r. The distal ends of the left curved wire 1611 and the right curved wire 161r are fixed to the distal end portion 111.

The bending portion 112 can be bent in a desired direction by pulling or relaxing the curved wire 160 (upper curved wire 161u, lower curved wire 161d, left curved wire 1611, right curved wire 161r), respectively.

The internal soft portion 119 is along and flexible tubular member. The curved wire 160, the channel tube 171 and the imaging cable 173 and the light guide 174 are inserted into the internal path 101 formed in the internal soft portion 119.

### [Stand 200]

The stand 200 is a self-standing stand that can be placed at a position away from the trolley 800. The stand 200 includes a pedestal 210, a pole 220, a support plate 230, and a connector 240.

The pedestal 210 is a leg for supporting the pole 220. The pedestal 210 illustrated in FIG. 1 is formed in a disk shape, but the pedestal 210 may have another shape as long as it can support the pole 220. The pole 220 is a support column that stands up from the pedestal 210 and supports the support plate 230. The pole 220 supports the support plate 230 and the connector 240 at a position higher than the operation table T.

The support plate 230 is provided on the upper part of the pole 220 and supports the connector 240. The support plate 230 is rotatably attached to the pole 220 in the horizontal direction. The connector 240 supported by the support plate 230 is rotatable in the horizontal direction.

The connector 240 detachably connects the drive cable 570 and the endoscope 100. Further, the video control cable 670 and the endoscope 100 are detachably connected to each other. The connector 240 has a forceps opening 250, a first connector 260, and a second connector 270.

The forceps opening 250 is an insertion port for inserting the treatment tool 400 into the channel tube 171 of the endoscope 100. The forceps opening 250 is provided in the first connector 260.

The first connector 260 is a connector to which the proximal end of the internal soft portion 119 of the endoscope 100 is attached. The first connector 260 is detachable from the second connector 270. Further, the first connector 260 is detachable from the distal end of the video control cable 670.

The second connector 270 is a connector to which the distal end of the drive cable 570 is attached. The second connector 270 is detachable from the first connector 260.

The mounted first connector 260 and second connector 270 can be installed on the support plate 230 as shown in FIG. 1. The first connector 260 and the second connector 270 are fixed so as not to be detachable by installing them on the support plate 230. The first connector 260 and the second connector 270 can be attached and detached by removing them from the support plate 230.

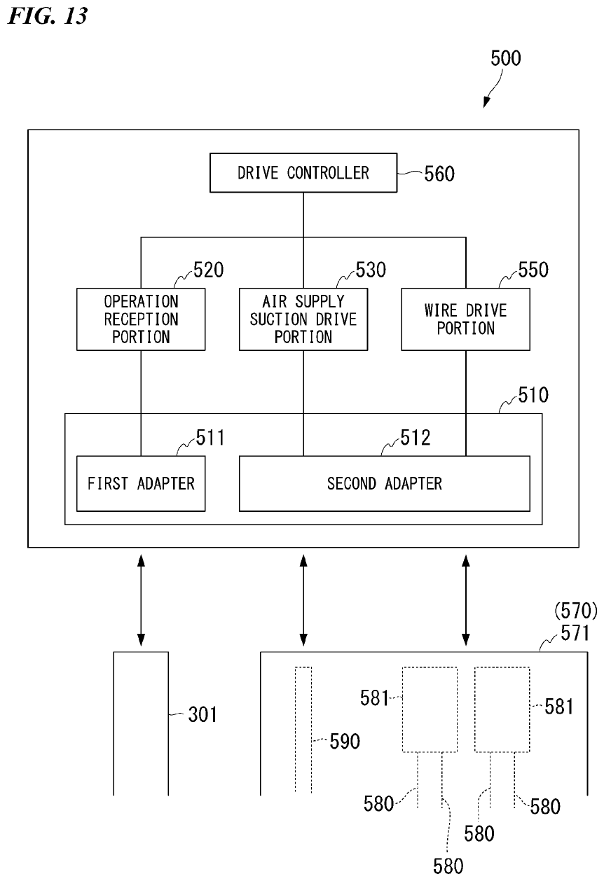

When the first connector 260 and the second connector 270 are attached, the drive wire 580 (see FIG. 13) of the drive cable 570 is connected so that the curved wire 160 of the endoscope 100 can be pulled and relaxed. Further, the air supply suction tube 590 (see FIG. 13) of the drive cable 570 is connected to the channel tube 171 of the endoscope 100.

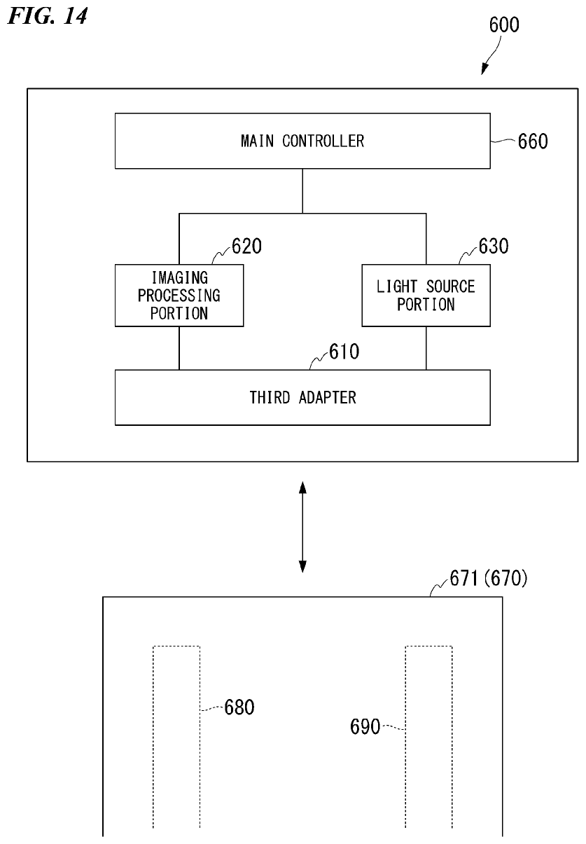

When the video control cable 670 is attached to the first connector 260, the video cable 680 (see FIG. 14) of the video control cable 670 is connected to the imaging cable 173 of the endoscope 100. Further, the light cable 690 (see FIG. 14) of the video control cable 670 is connected to the light guide 174 of the endoscope 100.

### [Operation Device 300]

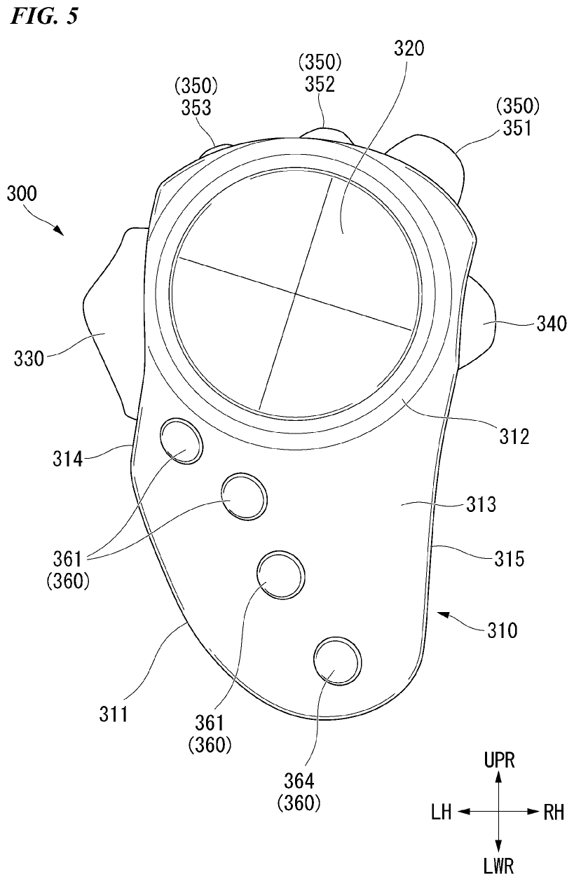

FIG. 5 is a diagram showing an operation device 300.

The operation device (controller) 300 is a device for inputting an operation (particularly, an operation for driving the endoscope 100) of a surgeon who controls the endoscope system 1000. The input operation input is transmitted to the drive device 500 or the like by wireless communication.

The operation device 300 includes an operation portion main body 310, a touch pad 320, a UD operation lever 330, an LR operation lever 340, a main operation button 350, and a sub operation button 360.

In the following description, the direction perpendicular to the touch pad 320 is defined as “front-back direction”, and the direction in which the touch pad 320 is provided with respect to the operation portion main body 310 is defined as “forward FR”, whose opposite direction is defined as “rear RR”. Further, the longitudinal direction of the operation portion main body 310 is defined as “vertical direction”, and the direction in which the touch pad 320 is attached to the operation portion main body 310 is defined as “upward UPR”, whose opposite direction is defined as “downward LWR”. The direction rightward toward the rear RR is defined as “right RH”, whose opposite direction is defined as “left LH”. The direction toward the right RH or the left LH is defined as “left-right direction”.

The operation portion main body 310 has a shape that can be held by the surgeon S with the left hand L, and is formed in an egg shape similar to a mouse, which is an input device of a PC. A grip 311 is formed on the lower LWR and the left LH of the operation portion main body 310. The grip 311 is a curved surface inclined in the vertical direction and the horizontal direction. The lower LWR of the operation portion main body 310 on which the grip 311 is formed is narrower than that of the upper UPR.

The front FR of the operation portion main body 310 is formed with a touch pad support portion 312 provided on the upper UPR and a front surface 313 provided on the lower LWR. The touchpad support portion 312 is formed in a circular shape and supports the touchpad 320.

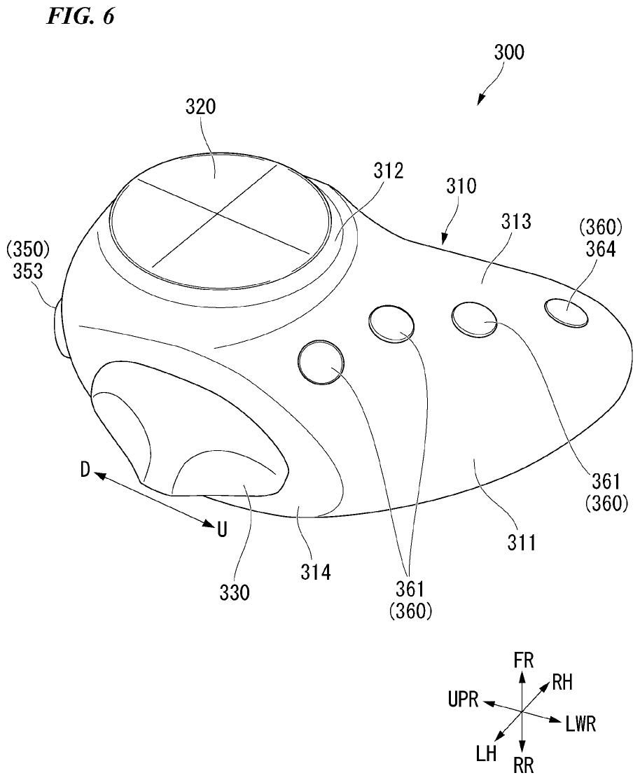

FIG. 6 is a diagram showing the operation device 300 as seen from the left LH.

A left side surface 314 facing the left LH is formed on the left LH of the operation portion main body 310. As shown in FIG. 5, the left side surface 314 is arranged adjacent to the touch pad support portion 312.

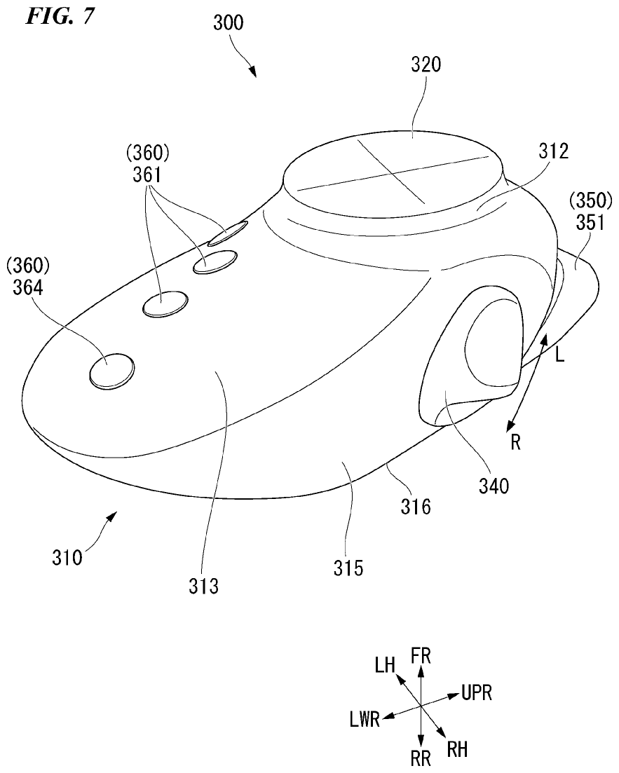

FIG. 7 is a diagram showing the operation device 300 as seen from the right RH.

A right side surface 315 facing the right RH is formed on the right RH of the operation portion main body 310. The right side surface 315 is a side surface larger than the left side surface 314.

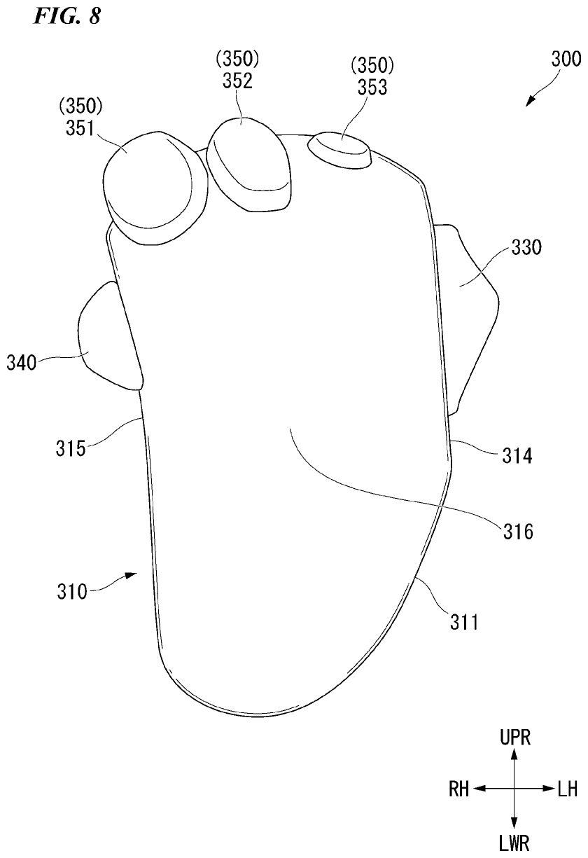

FIG. 8 is a diagram showing the operation device 300 as seen from the rear RR.

A dome-shaped rear surface 316 is formed on the rear RR of the operation portion main body 310. The surgeon S can bring the palm of the left hand L into contact with the posterior surface 316.

The touch pad 320 is a pointing device to which an operation for a bending portion 112 or the like is input. The touchpad 320 has a circular input area. The touch pad 320 outputs the two-dimensional coordinates of the touch pad 320 corresponding to the touched position in response to the operation input by touching the touch pad 320. The touch pad 320 is, for example, a resistance film type touch pad or a capacitive touch pad. Further, the touch pad 320 may be a touch panel provided with a liquid crystal display. The operation device 300 transmits the two-dimensional coordinates of the touch pad 320 to the drive device 500.

As shown in FIG. 6, the UD operation lever 330 is provided on the left side surface 314, and is a lever to which an operation of bending the bending portion 112 in the UD direction is input. The UD operation lever 330 is provided so as to be able to move forward and backward in the vertical direction. When the UD operation lever 330 is pushed into the upper UPR, the operation device 300 transmits an operation input for bending the bending portion 112 toward the lower side D to the drive device 500. When the UD operation lever 330 is pushed into the lower LWR, the operation device 300 transmits an operation input for bending the bending portion 112 toward the upper U to the drive device 500. The direction in which the bending portion 112 corresponding to the direction in which the UD operation lever 330 is pushed (upper UPR/lower LWR) is curved (lower D/upper U) may be reversed.

As shown in FIG. 7, the LR operation lever 340 is provided on the right side surface 315, and is a lever to which an operation of bending the bending portion 112 in the LR direction is input. The LR operation lever 340 is provided so as to be able to advance and retreat in the front-rear direction. When the LR operation lever 340 is pushed into the front FR, the operation device 300 transmits an operation input for bending the bending portion 112 toward the left side L to the drive device 500. When the UD operation lever 330 is pushed into the rear RR, the operation device 300 transmits an operation input for bending the bending portion 112 toward the right side R to the drive device 500. The direction in which the bending portion 112 corresponding to the direction in which the LR operation lever 340 is pushed (front FR/rear RR) is curved (left side L/right side R) may be reversed.

As shown in FIG. 5, the touch pad 320, the UD operation lever 330, and the LR operation lever 340 are arranged side by side from the left LH to the right RH when viewed from the front-rear direction.

The main operation button 350 is a button for inputting an operation for the main function of the endoscope 100. The main operation button 350 includes an air supply/water supply button 351, a suction button 352, and a release button 353. The main operation button 350 is attached to the upper UPR of the rear surface 316. The main operation button 350 is provided on the UPR above (upper) the center of the touch pad 320 in the vertical direction. As shown in FIGS. 5 and 8, the air supply/water supply button 351, the suction button 352, and the release button 353 are arranged side by side from the right RH to the left LH.

The air supply/water supply button 351 is attached to the upper UPR of the rear surface 316, and is a push button for inputting an operation of supplying air/water from the opening 111a of the distal end portion 111 of the endoscope 100. When the air supply/water supply button 351 is pushed in, the operation device 300 transmits an operation input for executing the air supply/water supply to the drive device 500.

The suction button 352 is attached to the upper UPR of the rear surface 316, and is a push button to which an operation of performing suction is input from the opening 111a of the distal end portion 111 of the endoscope 100. When the suction button 352 is pushed in, the operation device 300 transmits an operation input for performing suction to the drive device 500.

The release button 353 is attached to the upper UPR of the rear surface 316, and is a push button for inputting an operation of saving the captured image acquired by the video control device 600 from the imaging portion 111c of the endoscope 100. When the release button 353 is pressed, the operation device 300 transmits an operation input for storing the captured image to the drive device 500.

The sub operation button 360 has a programmable button 361 and a weakening button 364. The sub operation button 360 is attached to the front surface 313 of the operation portion main body 310. As shown in FIG. 5, the sub operation buttons 360 are arranged side by side from the upper left to the lower right. As a result, when the operation device 300 is held in the first position P1 or the second position P2, which will be described later, the thumb FT can easily reach any of the sub operation buttons 360.

The programmable button 361 is attached to the front 313 and is a push button to which an arbitrarily set operation is input. In this embodiment, three programmable buttons 361 are provided on the operation portion main body 310.

The weakening button 364 is attached to the front surface 313 and is a push button to which an operation of releasing the force for pulling the curved wire 160 of the endoscope 100 is input. When the weakening button 364 is pushed in, the operation device 300 transmits an operating input for releasing the force for pulling the curved wire 160 to the drive device 500. Alternatively, when the weakening button 364 is pushed in, an input for starting a control mode for controlling the bending portion 112 to bend according to an external force received from the surroundings may be transmitted to the drive device 500.

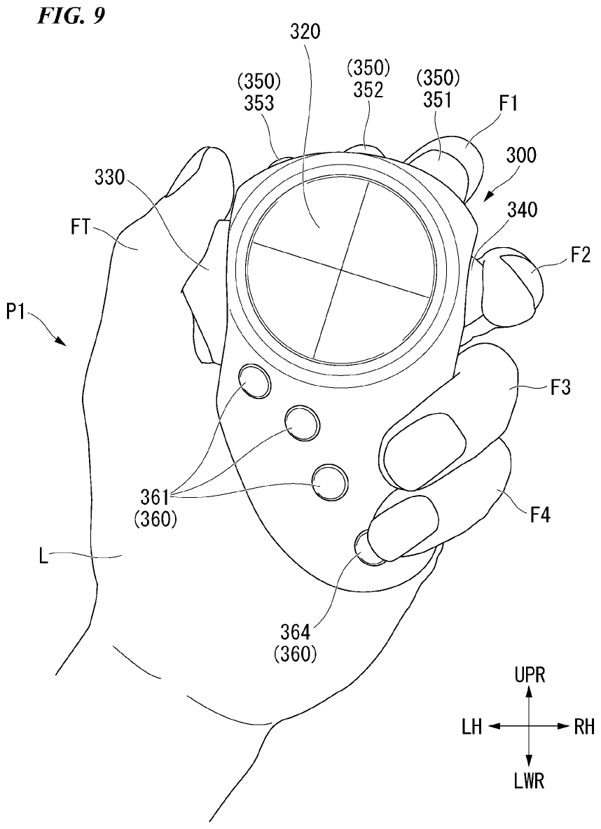

FIGS. 9 to 11 are views showing the operation device 300 held in the first position P1.

The first position P1 is a position in which the surgeon S holds the operation device 300 in order to operate the UD operation lever 330 and the LR operation lever 340 to bend the bending portion 112 of the endoscope 100. In the first position P1, the surgeon S brings the palm of the left hand L into contact with the rear surface 316, operates the UD operation lever 330 with the thumb FT, operates the LR operation lever 340 with the middle finger F2, and operates the main operation button 350 with the index finger F1.

The operation portion main body 310 is formed in an egg shape similar to a mouse, which is an input device of a PC. The surgeon S can firmly hold the operation device 300 simply by bringing the palm of the hand into contact with the rear surface 316, placing the operation portion main body 310 on the palm, and grasping the operation portion body 310. Therefore, the surgeon S is less likely to feel fatigue even if he/she continues to hold the operation device 300.

The UD operation lever 330 is provided on the left side surface 314, and the LR operation lever 340 is provided on the right side surface 315. Further, the UD operation lever 330 and the LR operation lever 340 are arranged side by side in the left-right direction when viewed from the front-rear direction. Therefore, the surgeon S can easily hold the UD operation lever 330 and the LR operation lever 340 by sandwiching them with the thumb and the middle finger F2.

When the UD operation lever 330 and the LR operation lever 340 are sandwiched and held by the thumb FT and the middle finger F2, the advance/retreat direction of the UD operation lever 330 substantially coincides with the movable direction of the first joint of the thumb FT, and the advancing/retreating direction of the LR operation lever 340 substantially coincides with the movable direction of the first joint and the second joint of the middle finger F2. That is, the UD operation lever 330 and the LR operation lever 340 have a structure that is anatomically easy to operate with the thumb FT and the middle finger F2. Therefore, the surgeon S is easy to operate when operating the bending portion 112, and is less likely to feel fatigue.

The operation feeling of operating the UD operation lever 330 with the thumb FT and the LR operation lever 340 with the middle finger F2 is similar to the operation feeling of operating the ankle knob of the operation portion of the existing flexible endoscope. Therefore, the surgeon S who is accustomed to the operation of the existing flexible endoscope can easily use the UD operation lever 330 and the LR operation lever 340 of the operation device 300.

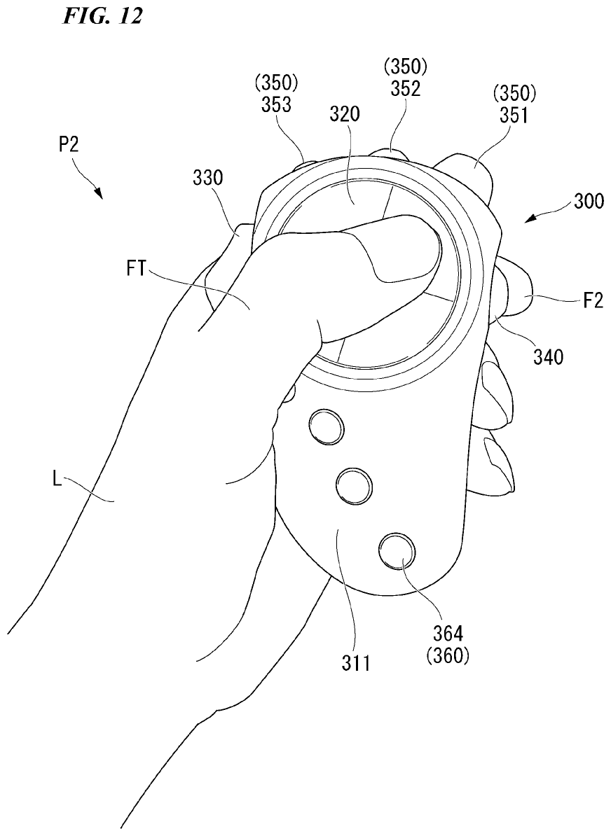

FIG. 12 is a diagram showing an operation device 300 held at the second position P2.

The second position P2 is a position in which the surgeon S holds the operation device 300 in order to operate the touch pad 320 to bend the bending portion 112 of the endoscope 100. In the second position P2, the surgeon S brings the palm of the left hand L into contact with the rear surface 316, operates the touch pad 320 with the thumb FT, and operates the main operation button 350 with the index finger F1.

The operation portion main body 310 is formed with a grip 311 on the lower LWR and the left LH of the operation portion main body 310. The surgeon S can arrange the thumb FT at a position where the touch pad 320 can be easily operated by fixing the base of the thumb FT to the grip 311.

The UD operation lever 330 and the touch pad 320 are arranged next to each other in the left-right direction. Therefore, the surgeon S can quickly change the position for holding the operation device 300 from the first position P1 to the second position P2 by moving the position of the thumb FT from the UD operation lever 330 to the touch pad 320. Further, the surgeon S can quickly change the position for holding the operation device 300 from the second position P2 to the first position P1 by moving the position of the thumb FT from the touch pad 320 to the UD operation lever 330.

The operation device 300 may be optimized for the shape and shape of the left hand L of the surgeon S according to the shape and size. The surgeon S may use his/her own operation device 300 optimized for his/her left hand L in combination with 1000 in the endoscope system.

### [Drive 500]

FIG. 13 is a functional block diagram of the drive device 500.

The drive device 500 includes an adapter 510, an operation reception portion 520, an air supply suction drive portion 530, a wire drive portion 550, and a drive controller 560.

The adapter 510 has a first adapter 511 and a second adapter 512. The first adapter 511 is an adapter to which the operation cable 301 is detachably connected. The second adapter 512 is an adapter to which the connecting portion 571 provided on the proximal end side of the drive cable 570 is detachably connected. The drive cable 570 includes a drive wire 580 and an air supply suction tube 590.

The operation reception portion 520 has a known wireless reception module, and receives an operation input from the operation device 300 by wireless communication. The operation reception portion 520 may receive an operation input from the operation device 300 via the operation cable 301.

The air supply suction drive portion 530 is connected to the air supply suction tube 590 of the drive cable 570. The air supply suction drive portion 530 includes a pump or the like, and supplies air or liquid to the air supply suction tube 590. Further, the air supply suction drive portion 530 sucks air from the air supply suction tube 590. The water supply is not limited to the delivery of water into the body, and may include, for example, the delivery of a chemical solution or the like into the body.

The wire drive portion 550 is connected to the drive wire 580 of the drive cable 570 via the drive wire adapter 581. The wire drive portion 550 has a drive portion and an encoder (not shown). The drive portion pulls or relaxes the drive wire 580 of the drive cable 570 by a pulley or the like. The encoder detects the traction amount of the drive wire 580. The detection result of the encoder is acquired by the drive controller 560 of the drive device 500.

The drive controller 560 controls the entire drive device 500. The drive controller 560 acquires the operation input received by the operation reception portion 520. The drive controller 560 controls the air supply suction drive portion 530 and the wire drive portion 550 based on the acquired operation input. When the acquired operation input is an operation input related to the video control device 600, the drive controller 560 transmits the acquired operation input to the main controller 660 of the video control device 600.

The drive controller 560 controls the wire drive portion 550 based on the acquired operation input. When the drive controller 560 controls the wire drive portion 550 based on the operation input from the touch pad 320, the drive controller 560 controls so that the distance (input sensitivity, resolution) at which the bending portion 112 bends with respect to a predetermined operation input becomes smaller, which is hereafter also referred to as “fine movement control”. On the other hand, when controlling the wire drive portion 550 based on the operation inputs from the UD operation lever 330 and the LR operation lever 340, the drive controller 560 controls so that the distance (input sensitivity, resolution) at which the bending portion 112 bends with respect to a predetermined operation input is larger than that of the fine movement control, which is hereinafter also referred to as “coarse movement control”.

The drive controller 560 is a computer capable of executing a program including a processor, a memory, a storage portion capable of storing programs and data, and an input/output control portion. The function of the drive controller 560 is realized by the processor executing the program. At least some functions of the drive controller 560 may be realized by a dedicated logic circuit.

The drive controller 560 may further have a configuration other than the processor, the memory, the storage portion, and the input/output control portion. For example, the drive controller 560 may further have an image calculation portion that performs a part or all of image processing and image recognition processing. By further having an image calculation portion, the drive controller 560 can execute specific image processing and image recognition processing at high speed. The image calculation portion may be mounted on a separate hardware device connected by a communication line.

### [Video Control Device 600]

FIG. 14 is a functional block diagram of the video control device 600.

The video control device 600 includes a third adapter 610, an imaging processing portion 620, a light source portion 630, and a main controller 660.

The third adapter 610 is an adapter to which the connecting portion 671 provided on the proximal end side of the video control cable 670 is detachably connected. The video control cable 670 includes a video cable 680 and a light cable 690.

The imaging processing portion 620 is connected to the video cable 680. The imaging processing portion 620 converts the imaging signal acquired from the imaging portion 111c of the distal end portion 111 into a captured image via the imaging cable 173 and the video cable 680.

The light source portion 630 is connected to the light cable 690. The light source portion 630 generates illumination light to be applied to the imaging target. The illumination light generated by the light source portion 630 is guided to the illumination portion 111b of the distal end portion 1 via the light cable 690 and the light guide 174.

The main controller 660 is a computer capable of executing a program equipped with a processor, memory, and the like. The function of the main controller 660 is realized by the processor executing the program. At least some functions of the main controller 660 may be realized by a dedicated logic circuit.

The main controller 660 can perform image processing on the captured image acquired by the imaging processing portion 620. The main controller 660 can generate a GUI image or a CG image for the purpose of providing information to the surgeon S. The main controller 660 can display the captured image, GUI image, and CG image on the display device 900.

When the main controller 660 detects the operation input when the release button 353 is pressed, the main controller 660 saves the captured image acquired by the imaging processing portion 620.

The main controller 660 is not limited to an integrated hardware device. For example, the main controller 660 may be configured by partially separating it as a separate hardware device and then connecting the separated hardware device via a communication line. For example, the main controller 660 may be a cloud system in which the separated storage portions 563 are connected by a communication line.

The main controller 660 may further have a configuration other than the processor and the memory. For example, the main controller 660 may further have an image calculation portion that performs a part or all of the image processing and the image recognition processing performed by the processor. By further having an image calculation portion, the main controller 660 can execute specific image processing and image recognition processing at high speed. The image calculation portion may be mounted on a separate hardware device connected by a communication line.

### [Operation of Endoscope System 1000]

Next, the operation of the endoscope system 1000 of this embodiment will be described. Specifically, a procedure for observing and treating an affected area formed on a tube wall in the large intestine using the endoscope system 1000 will be described.

The surgeon S inserts the insertion portion 110 of the endoscope 100 into the large intestine from the anus of the patient P from the distal end. As shown in FIG. 2, the surgeon S moves the insertion portion 110 while observing the captured image displayed on the display device 900 and operating the internal soft portion 119 with the right hand R to bring the distal end portion 111 closer to the affected area. Further, the surgeon S operates the operation device 300 held at the first position P1 to bend the bending portion 112 as necessary.

When the UD operation lever 330 and the LR operation lever 340 of the operation device 300 held in the first position P1 are operated, the bending portion 112 of the endoscope 100 is coarsely controlled (coarse movement control), so that it is easy for the surgeon S to perform the insertion operation and observation operation of the endoscope 100.

When treating the affected area, the surgeon S changes the position for holding the operation device 300 to the second position P2. When the touch pad 320 of the operation device 300 held in the second position P2 is operated, the bending portion 112 of the endoscope 100 is finely controlled (fine movement control), so that it is easy for the surgeon S to perform procedures that require precise operation of the endoscope 100 such as ESD (endoscopic submucosal dissection).

After the treatment of the affected area is completed, the surgeon S changes the position for holding the operation device 300 to the first position P1. When the UD operation lever 330 and the LR operation lever 340 of the operation device 300 held in the first position P1 are operated, the bending portion 112 of the endoscope 100 is coarsely controlled (coarse movement control), so that it is easy for the surgeon S to perform the removal operation of the endoscope 100.

According to the endoscope system 1000 according to the present embodiment, the operation device 300 to which the operation of the surgeon S is input can be easily handled and intuitively operated. The surgeon S can easily change the operation system of the operation device 300 by changing (first position P1 and second position P2) at the position where the operation device 300 is held.

Although the first embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Second Embodiment

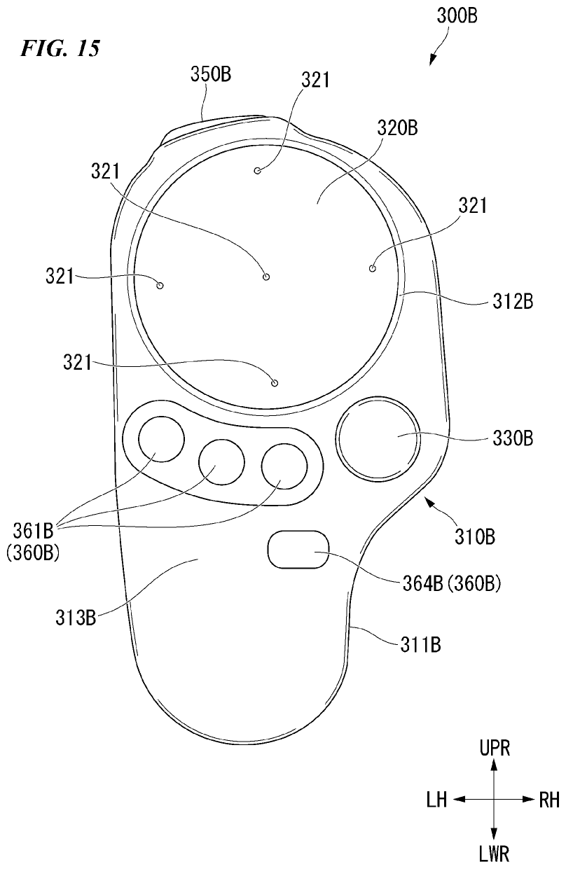

A second embodiment of the present invention will be described with reference to FIGS. 15 to 19. The endoscope system according to the second embodiment differs from the endoscope system 1000 according to the first embodiment only in the operation device 300. In the following description, the same reference numerals will be given to the configurations common to those already described, and duplicate description will be omitted.

The endoscope system according to the present embodiment includes an endoscope 100, a stand 200, an operation device 300B, a treatment tool 400, a drive device 500, an video control device 600, a trolley 800, and a display device 900.

FIG. 15 is a diagram showing an operation device 300B.

The operation device (controller) 300B is a device for inputting the operation of the surgeon who controls the endoscope system (particularly, the operation for driving the endoscope 100). The input operation input is transmitted to the drive device 500 or the like by wireless communication.

The operation device 300B includes an operation portion main body 310B, a touch pad 320B, a joystick 330B, a main operation button 350B, and a sub operation button 360B.

The operation portion main body 310B is formed in a shape that can be held by the surgeon S with the left hand L. A grip 311B is formed on the lower LWR and the right RH of the operation portion main body 310B. The lower LWR of the operation portion main body 310B on which the grip 311B is formed is narrower than that of the upper UPR.

The front FR of the operation portion main body 310B is formed with a touch pad support portion 312B provided on the upper UPR and a front surface 313B provided on the lower LWR. The touchpad support portion 312B is formed in a circular shape and supports the touchpad 320B.

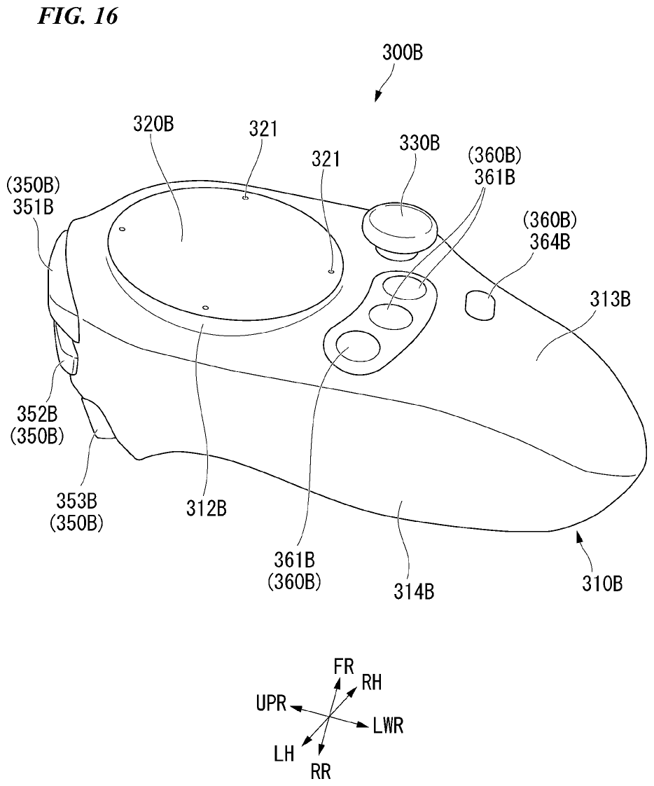

FIG. 16 is a diagram showing an operation device 300B seen from the left LH.

A left side surface 314B facing the left LH is formed on the left LH of the operation portion main body 310B. The surgeon S can bring the palm of the left hand L into contact with the left side surface 314B.

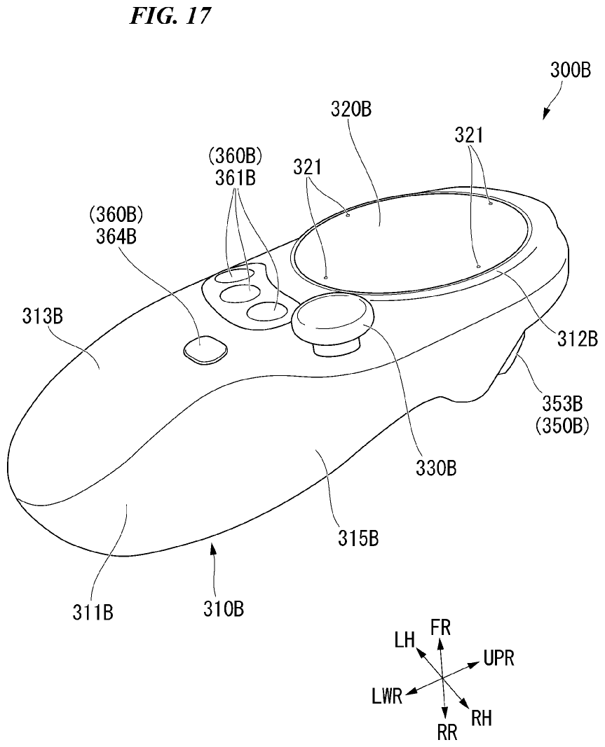

FIG. 17 is a diagram showing an operation device 300B seen from the right RH.

A right side surface 315B facing the right RH is formed on the right RH of the operation portion main body 310B. The lower LWR of the right side surface 315B forms part of the grip 311B.

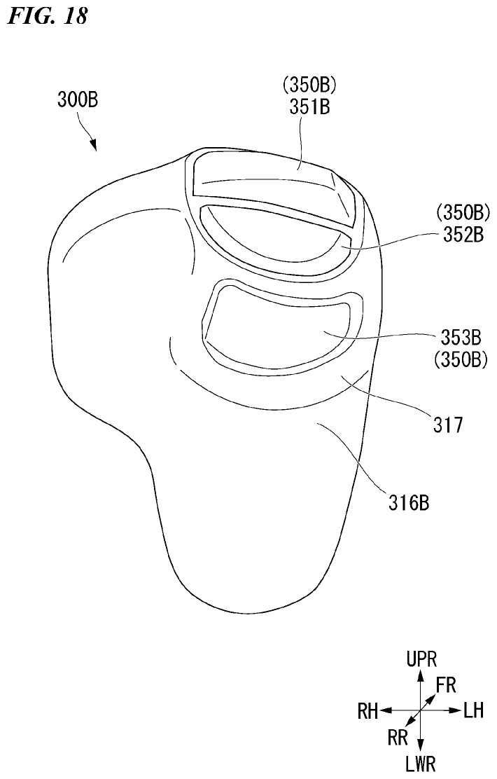

FIG. 18 is a diagram showing an operation device 300B seen from the rear RR.

The rear RR of the operation portion main body 310B is formed on the rear surface 316B. A button support portion 317 in which the main operation button 350B is arranged is formed on the upper UPR of the rear surface 316B. The button support portion 317 is a convex portion that protrudes to the rear RR.

The touch pad 320B is a pointing device to which an operation for the bending portion 112 or the like is input, like the touch pad 320 of the first embodiment. The touchpad 320B has a circular input area. The touch pad 320B outputs the two-dimensional coordinates of the touch pad 320B corresponding to the touched position in response to the operation input by touching the touch pad 320B. The operation device 300B transmits the two-dimensional coordinates of the touch pad 320B to the drive device 500.

The touch pad 320B has a convex portion 321 near the center and the upper, lower, left, and right ends. By confirming the convex portion 321 by tactile sensation, the surgeon S can recognize the position of the thumb FT on the touch pad 320B without visually confirming it.

The joystick 330B is a device to which an operation for the bending portion 112 or the like is input. The joystick 330B is attached to the front surface 313B, and is provided adjacent to the touch pad 320B at the lower right of the touch pad 320B. The operation device 300B transmits a directional operation with respect to the joystick 330B to the drive device 500.

The main operation button 350B is a button for inputting operations for the main functions of the endoscope 100. The main operation button 350B has an air supply/water supply button 351B, a suction button 352B, and a release button 353B. The main operation button 350B is attached to the button support portion 317 of the rear surface 316. The main operation button 350B is provided on the UPR above (upper) the center of the touch pad 320B in the vertical direction. As shown in FIGS. 16 and 18, the air supply/water supply button 351B, the suction button 352B, and the release button 353B are arranged side by side from the front FR toward the rear RR.

The air supply/water supply button 351B is attached to the button support portion 317 of the rear surface 316B, and is a push button to which an operation of supplying air/water is input from the opening 111a of the distal end portion 111 of the endoscope 100. When the air supply/water supply button 351B is pushed in, the operation device 300B transmits an operation input for executing the air supply/water supply to the drive device 500.

The suction button 352B is attached to the button support portion 317 of the rear surface 316B, and is a push button to which an operation of performing suction is input from the opening 111a of the distal end portion 111 of the endoscope 100. When the suction button 352B is pushed in, the operation device 300B transmits an operation input for performing suction to the drive device 500.

The release button 353B is attached to the button support portion 317 of the rear surface 316B, and is a push button for inputting an operation of saving the captured image acquired by the video control device 600 from the imaging portion 111c of the endoscope 100. When the release button 353B is pushed in, the operation device 300B transmits an operation input for storing the captured image to the drive device 500.

The sub operation button 360B has a programmable button 361B and a weakening button 364B. The sub operation button 360B is attached to the front surface 313B of the operation portion main body 310B.

The programmable button 361B is attached to the front 313B and is a push button to which an arbitrarily set operation is input. In this embodiment, three programmable buttons 361B are provided on the operation portion main body 310B.

The weakening button 364B is attached to the front surface 313B and is a push button to which an operation of releasing the force for pulling the curved wire 160 of the endoscope 100 is input. When the weakening button 364B is pushed in, the operation device 300B transmits an operating input for releasing the force for pulling the curved wire 160 to the drive device 500.

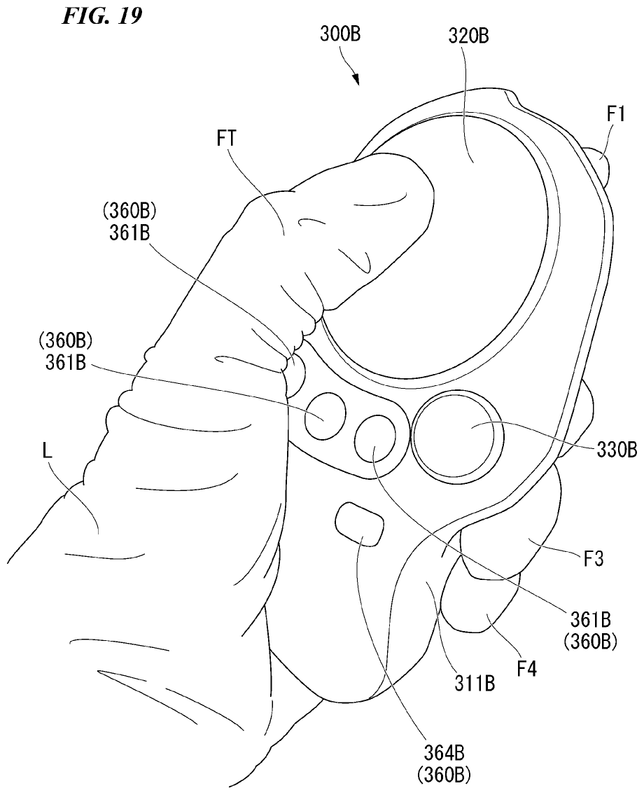

FIG. 19, is a diagram showing an operation device 300B held by the left hand L of the surgeon S.

The surgeon S brings the palm of his left hand L into contact with the left side surface 314B, operates the touch pad 320B and the joystick 330B with the thumb FT, and operates the main operation button 350B with the index finger F1 or the middle finger F2.

The surgeon S can stably hold the operation portion main body 310B by hooking the ring finger F3 and the little finger F4 on the grip 311B regardless of whether the thumb FT is placed on the touch pad 320B or the joystick 330B.

The touch pad 320B and the joystick 330B are provided adjacent to each other on the front surface 313B. Therefore, the surgeon S can easily change the operation input device (touch pad 320B, joystick 330B) that controls the bending portion 112.

The drive controller 560 controls the wire drive portion 550 based on the acquired operation input. When controlling the wire drive portion 550 based on the operation input from the joystick 330B, the drive controller 560 controls so that the distance (input sensitivity, resolution) at which the bending portion 112 bends with respect to a predetermined operation input becomes smaller (fine movement control). On the other hand, when controlling the wire drive portion 550 based on the operation input from the touch pad 320B, the drive controller 560 controls so that the distance (input sensitivity, resolution) at which the bending portion 112 bends with respect to a predetermined operation input is larger than that of the fine movement control (coarse movement control).

When the touch pad 320B of the operation device 300B is operated, the bending portion 112 of the endoscope 100 is coarsely controlled (coarse movement control), so that the surgeon S can easily perform an insertion operation, an observation operation, and a removal operation of the endoscope 100.

When the joystick 330B of the operation device 300B is operated, the bending portion 112 of the endoscope 100 is finely controlled (fine movement control), so that the surgeon S can easily perform a procedure that requires a precise operation of the endoscope 100 such as ESD (endoscopic submucosal dissection).

According to the endoscope system according to the present embodiment, the operation device 300B to which the operation of the surgeon S is input is easy to handle and can be operated intuitively. The surgeon S can easily change the operation system of the operation device 300 by changing the operation input device (touch pad 320B, joystick 330B) that operates the bending portion 112.

Although the second embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Third Embodiment

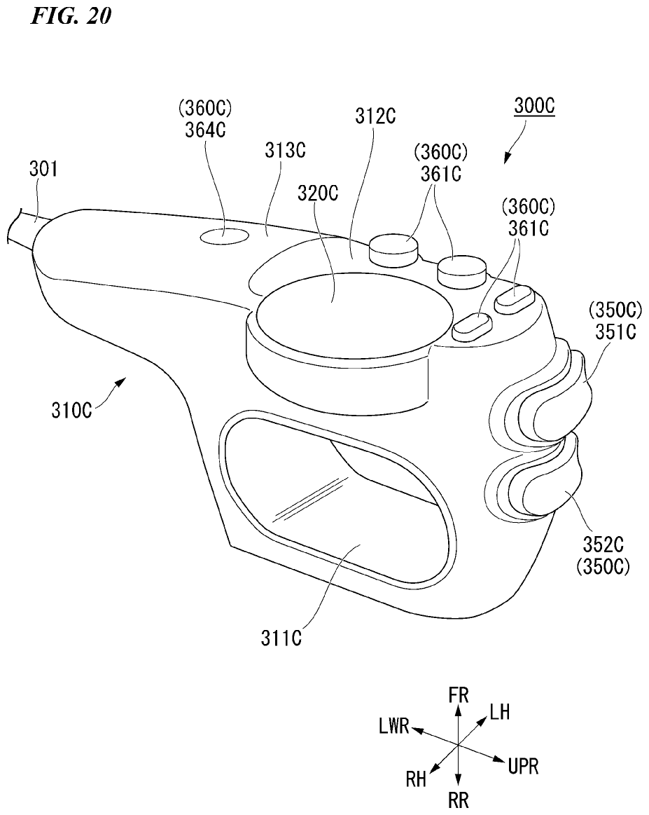

A third embodiment of the present invention will be described with reference to FIGS. 20 to 23. The endoscope system according to the third embodiment differs from the endoscope system 1000 according to the first embodiment only in the operation device 300. In the following description, the same reference numerals will be given to the configurations common to those already described, and duplicate description will be omitted.

The endoscope system according to the present embodiment includes an endoscope 100, a stand 200, an operation device 300C, a treatment tool 400, a drive device 500, an video control device 600, a trolley 800, and a display device 900.

FIG. 20 is a diagram showing an operation device 300C

The operation device (controller) 300C is a device for inputting the operation of the surgeon who controls the endoscope system (particularly, the operation for driving the endoscope 100). The input operation input is transmitted to the drive device 500 or the like via the operation cable 301.

The operation device 300C includes an operation portion main body 310C, a touch pad 320C, a main operation button 350C, and a sub operation button 360C.

The operation portion main body 310C is formed in a shape that can be held by the surgeon S with the left hand L. A handle 311C is formed on the rear RR of the operation portion main body 310.

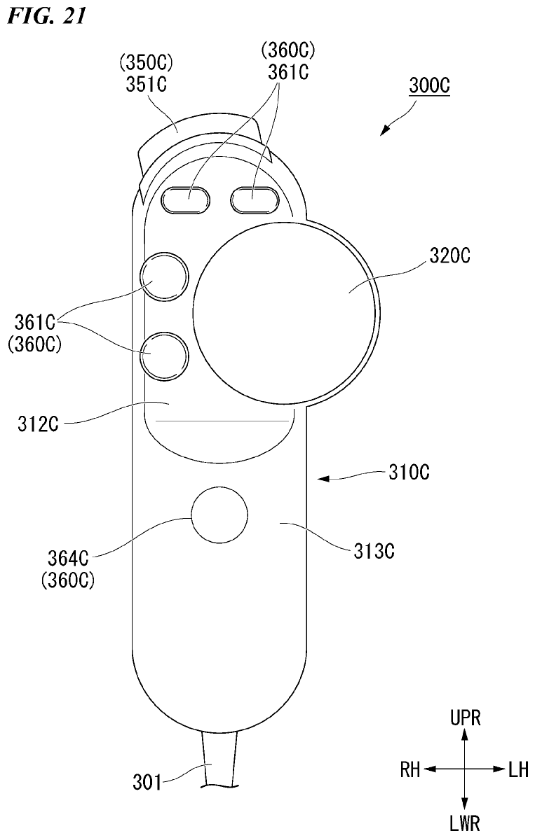

FIG. 21 is a diagram showing an operation device 300C seen from the front FR.

The front FR of the operation portion main body 310C is formed with an operation input support portion 312C provided on the upper UPR and a front surface (grip) 313B provided on the lower LWR The operation input support portion 312C supports the touch pad 320C and the programmable button 361C.

The touch pad 320C is a pointing device to which an operation for the bending portion 112 or the like is input, like the touch pad 320 of the first embodiment.

The main operation button 350C is a button for inputting operations for the main functions of the endoscope 100. The main operation button 350C has an air supply/water supply button 351C and a suction button 352C. The main operation button 350C is attached to the upper part of the operation portion main body 310C. The main operation button 350 is provided above (upper) UPR above the touch pad 320C in the vertical direction. The air supply/water supply button 351C and the suction button 352C are arranged side by side from the front FR toward the rear RR.

The air supply/water supply button 351C is a push button to which an operation for air supply/water supply is input, similar to the air supply/water supply button 351 of the first embodiment. The air supply/water supply button 351C is provided on the upper left of the operation portion main body 310C when viewed from the front-rear direction.

The suction button 352C is a push button to which an operation for performing suction is input, similar to the suction button 352 of the first embodiment. The suction button 352C is provided on the upper left of the operation portion main body 310C when viewed from the front-rear direction.

The sub operation button 360C has a programmable button 361C and a weakening button 364C.

The programmable button 361C is attached to the operation input support portion 312C and is a push button on which an arbitrarily set operation is input. In this embodiment, four programmable buttons 361C are provided on the operation portion main body 310C.

The programmable button 361C may be assigned as a button for switching a method (fine movement control or coarse movement control) in which the drive controller 560 controls the wire drive portion 550 based on the operation input from the touch pad 320C.

The weakening button 364C is attached to the front surface 313C, and is a push button to which an operation of releasing the force for pulling the curved wire 160 is input, similar to the weakening button 364 of the first embodiment.

According to the endoscope system according to the present embodiment, the operation device 300C to which the operation of the surgeon S is input can be easily handled and intuitively operated. As shown in FIG. 21, the touch pad 320C and the programmable button 361C are collectively arranged on the operation input support portion 312C provided in the upper UPR of the operation portion main body 310C, and it is easy to operate with the thumb FT of the left hand L of the surgeon S. Further, the air supply/water supply button 351C and the suction button 352C are provided on the upper left of the operation portion main body 310C when viewed from the front-rear direction, and can be easily operated by the index finger F1 or the middle finger F2 of the left hand L of the surgeon S. Further, the surgeon S does not need to always hold the operation device 300C if the left hand L is inserted through the handle 311C.

Although the third embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

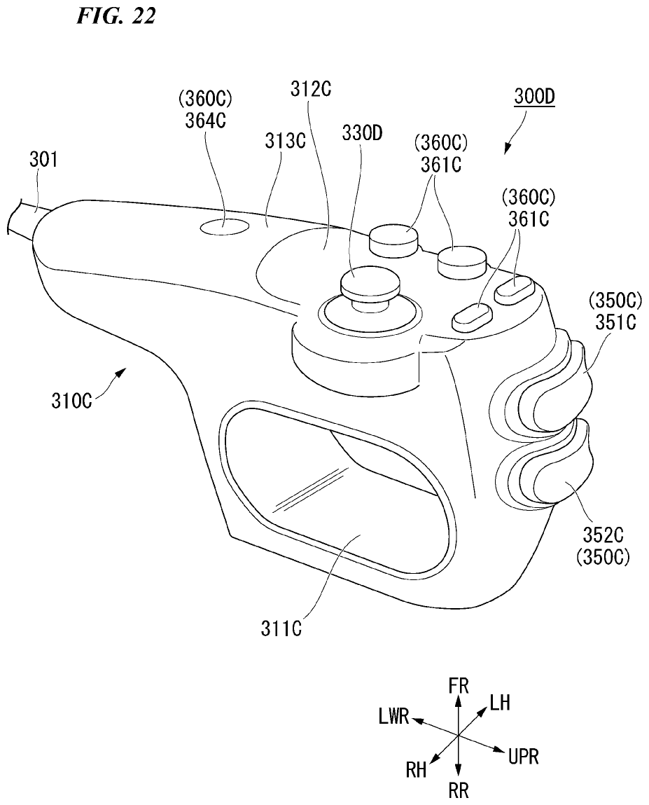

FIG. 22 is a diagram showing an operation device 300D which is a modification of the operation device 300C.

The operation device 300D is equipped with a joystick 330D instead of the touch pad 320C. The joystick 330D has the same function as the joystick 330B of the second embodiment.

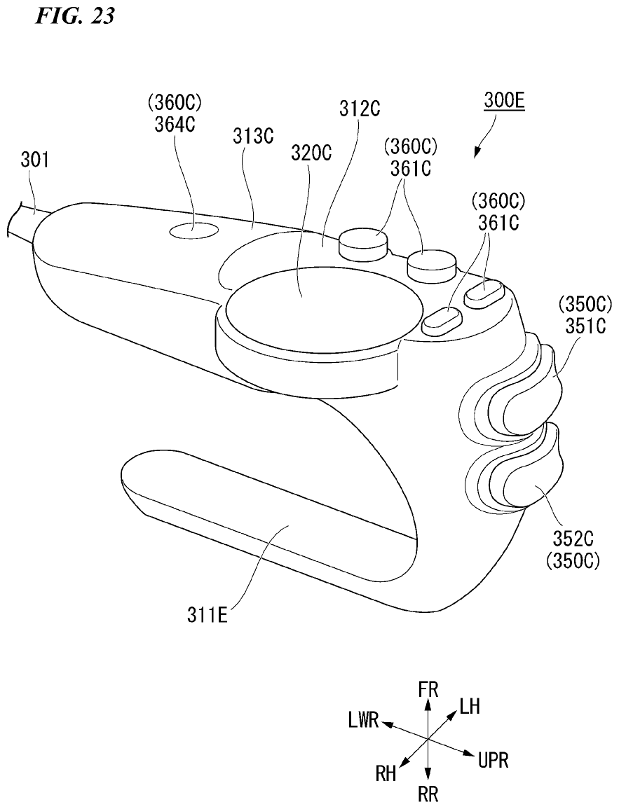

FIG. 23 is a diagram showing an operation device 300E which is a modification of the operation device 300C.

The operation device 300E is provided with a hanger 311E instead of the handle 311C. In the hanger 311E, the lower LWR in the vertical direction is open, and the surgeon S can easily insert the left hand L. Further, the surgeon S can hook the hanger 311E on a fixing device or the like to fix the operation device 300E when the surgeon S wants to separate the left hand L from the operation device 300E during the operation or when the surgeon S wants to store the operation device 300E before and after the operation.

### Fourth Embodiment

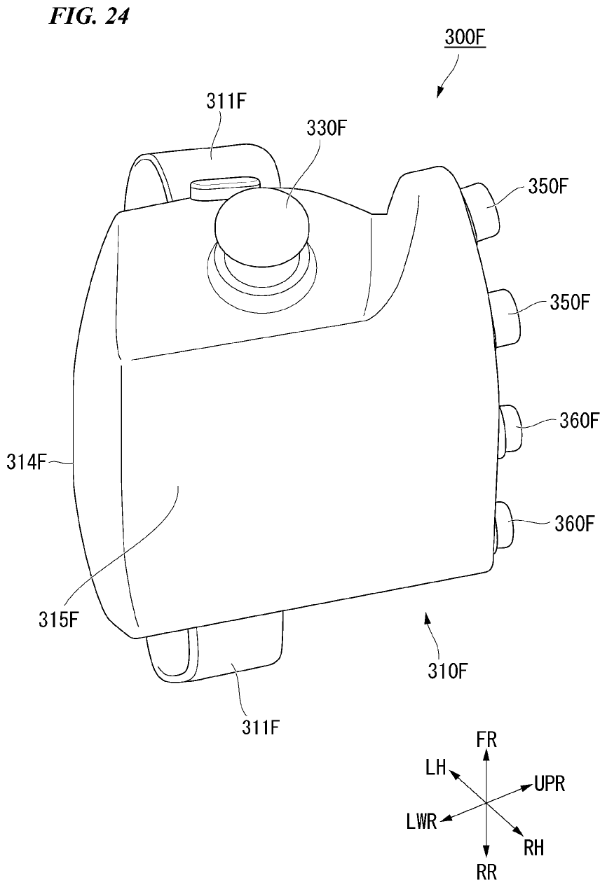

A fourth embodiment of the present invention will be described with reference to FIG. 24. The endoscope system according to the fourth embodiment differs from the endoscope system 1000 according to the first embodiment only in the operation device 300. In the following description, the same reference numerals will be given to the configurations common to those already described, and duplicate description will be omitted.

The endoscope system according to the present embodiment includes an endoscope 100, a stand 200, an operation device 300F, a treatment tool 400, a drive device 500, an video control device 600, a trolley 800, and a display device 900.

FIG. 24 is a diagram showing an operation device 300F.

The operation device 300F includes an operation portion main body 310F, a joystick 330F, a main operation button 350F, and a sub operation button 360F.

The operation portion main body 310F is formed in a substantially plate shape having a left side surface 314F and a right side surface 315F. A belt 311F is attached to the left side surface 314F, and the surgeon S can stably hold the operation portion main body 310F by bringing the palm of the left hand L into contact with the left side surface 314F and attaching the belt 311F to the left hand L.

The joystick 330F is provided on the right side surface 315F at a position where the operation portion main body 310F is held and easily operated by the thumb FT of the left hand L.

The main operation button 350F and the sub operation button 360F are arranged side by side from the front FR toward the rear RR in the upper part of the operation portion main body 310F. The main operation button 350F and the sub operation button 360F are provided at positions that are easy to operate with the index finger F1 or the middle finger F2 of the left hand L of the surgeon S.

According to the endoscope system according to the present embodiment, the operation device 300F to which the operation of the surgeon S is input can be easily handled and intuitively operated. The surgeon S does not need to always hold the operation device 300F if the left hand L is inserted through the belt 311F.

Although the fourth embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Fifth Embodiment

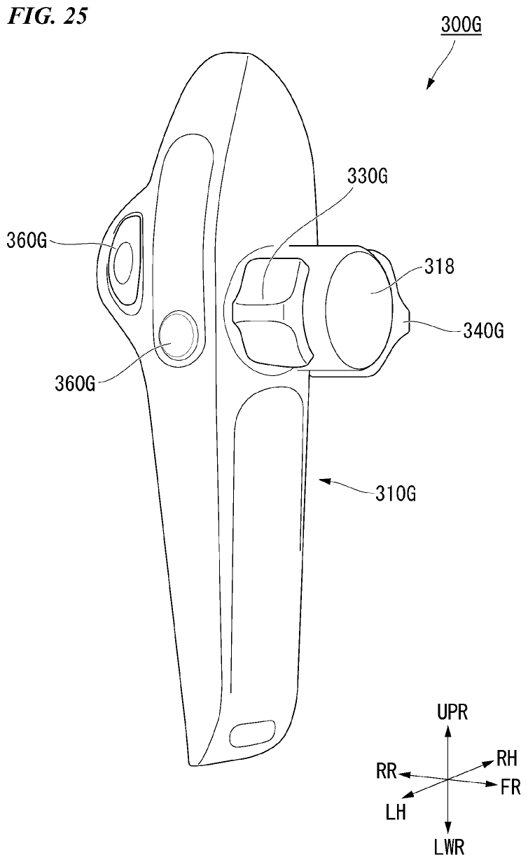

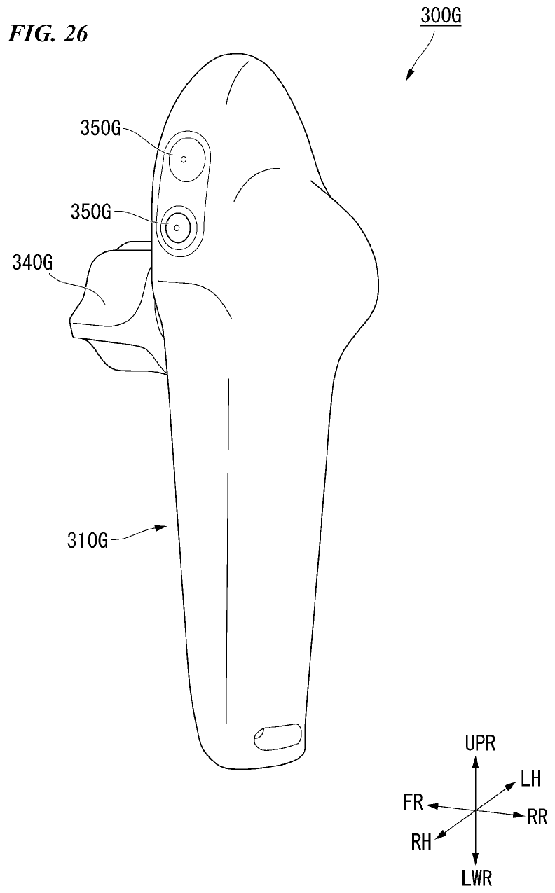

A fifth embodiment of the present invention will be described with reference to FIGS. 25 to 26. The endoscope system according to the fifth embodiment differs from the endoscope system 1000 according to the first embodiment only in the operation device 300. In the following description, the same reference numerals will be given to the configurations common to those already described, and duplicate description will be omitted.

The endoscope system according to the present embodiment includes an endoscope 100, a stand 200, an operation device 300G, a treatment tool 400, a drive device 500, an video control device 600, a trolley 800, and a display device 900.

FIGS. 25 and 26 are views showing the operation device 300G.

The operation device 300G includes an operation portion main body 3100, a UD operation lever 330G, an LR operation lever 340G, a main operation button 3500, and a sub operation button 360G.

The operation portion main body 310G is formed in a substantially cylindrical shape that can be held by the surgeon S with the left hand L. A main operation button 350G is provided on the right RH of the operation portion main body 310G. A sub operation button 360G is provided on the left LH of the operation portion main body 310G.

The operation portion main body 310G is provided with a columnar knob 318 protruding from the front FR. A UD operation lever 330G is provided on the left LH of the knob 318. An LR operation lever 340G is provided on the right RH of the knob 318.

The UD operation lever 330G is a lever to which an operation of bending the bending portion 112 in the UD direction is input, similar to the UD operation lever 330 of the first embodiment. The UD operation lever 330 is provided so as to be able to advance and retreat along the outer periphery of the knob 318.

The LR operation lever 340G is a lever to which an operation of bending the bending portion 112 in the LR direction is input, similar to the LR operation lever 340 of the first embodiment. The LR operation lever 340 is provided so as to be able to advance and retreat along the outer circumference of the knob 318.

The operation feeling of operating the UD operation lever 330G with the thumb FT and the LR operation lever 340G with the middle finger F2 is similar to the operation feeling of operating the ankle knob of the operation portion of the existing flexible endoscope. Therefore, the surgeon S who is accustomed to the operation of the existing flexible endoscope can easily use the UD operation lever 330G and the LR operation lever 340G of the operation device 300G.

According to the endoscope system according to the present embodiment, the operation device 300G to which the operation of the surgeon S is input is easy to handle and can be operated intuitively. Since the UD operation lever 330G and the LR operation lever 340G require less finger movement for operation input than the ankle knob of the existing flexible endoscope, the burden on the operation input of the surgeon S can be reduced.

Although the fifth embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

Although the procedure in which the treatment target is the large intestine is described in detail in the above embodiment, the treatment target of the procedure using the endoscope system is not limited to this. The procedure using the endoscopic system can target any body cavity including the upper gastrointestinal tract, the lower gastrointestinal tract, and the like.

What is claimed is:

1. An endoscope system comprising:
an endoscope including a bending portion that is capable of bending;
a drive device that is detachably connected to the endoscope and drives the endoscope; and
an operation device that is communicably connected to the drive device and has an input portion that inputs an operation for driving the endoscope,
wherein the drive device can select a method of controlling the bending portion with respect to the operation input from the input portion from fine movement control and coarse movement control, and
a distance at which the drive device bends the bending portion by the coarse movement control with respect to a predetermined operation input is larger than a distance at which the drive device bends the bending portion by the fine movement control.

2. The endoscope system according to claim 1, wherein
the operation device has a coarse movement input portion and a fine movement input portion as the input portion,
the drive device performs the coarse movement control of the bending portion based on the operation input from the coarse movement input portion, and
the drive device performs the fine movement control of the bending portion based on the operation input from the fine movement input portion.

3. The endoscope system according to claim 2, wherein
the coarse movement input portion is a lever, and
the fine movement input portion is a touch pad.

4. The endoscope system according to claim 2, wherein the coarse movement input portion is arranged on both sides of the fine movement input portion.

5. The endoscope system according to claim 4, wherein
the coarse movement input portion includes
a vertical operation lever that bends the bending portion in vertical direction, and
a left-right operation lever that bends the bending portion in left-right direction,
the vertical operation lever is provided on a left side surface of the operation device, and
the left-right operation lever is provided on a right side surface of the operation device.

6. The endoscope system according to claim 2, wherein the coarse movement input portion and the fine movement input portion are arranged side by side in left-right direction perpendicular to a longitudinal direction of the operation device.

7. The endoscope system according to claim 6, wherein
the operation device includes an air supply/water supply button and a suction button as the input portion,
the drive device controls air supply/water supply of the endoscope based on the operation input from the air supply/water supply button,
the drive device controls suction of the endoscope based on the operation input from the suction button, and
the air supply/water supply button and the suction button are arranged side by side in the left-right direction.

8. The endoscope system according to claim 2, wherein
the coarse movement input portion is a touch pad, and
the fine movement input portion is a joystick.

9. The endoscope system according to claim 8, wherein the coarse movement input portion is arranged adjacent to the fine movement input portion.

10. The endoscope system according to claim 8, wherein the coarse movement input portion is provided on upper side in a vertical direction along a longitudinal direction of the operation device.

11. The endoscope system according to claim 6, wherein
the operation device has an air supply/water supply button and a suction button as the input portion,
the drive device controls air supply/water supply of the endoscope based on the operation input from the air supply/water supply button,
the drive device controls suction of the endoscope based on the operation input from the suction button, and
the air supply/water supply button and the suction button are arranged side by side in a vertical direction along a longitudinal direction of the operation device.

12. The endoscope system according to claim 1, wherein the drive device selects a method to control the bending portion from the fine movement control and the coarse movement control based on the operation input from a button of the operation device.

13. A controller that controls an endoscope including a bending portion that is capable of bending,
the controller including a coarse movement input portion and a fine movement input portion, which are input portion that controls the bending portion,
wherein the fine movement input portion and the coarse movement input portion are different types of input portion.

14. The controller according to claim 13, wherein
the coarse movement input portion is a lever, and
the fine movement input portion is a touch pad.

15. The controller according to claim 13, wherein
the coarse movement input portion is a touch pad, and
the fine movement input portion is a joystick.

16. A controller that controls an endoscope including a bending portion that is capable of bending,
the controller including a bending control input portion that controls the bending portion, an air supply/water supply button that controls air supply or water supply of the endoscope, and a suction button that controls suction of the endoscope,
the air supply/water supply button and the suction button are provided above center of the bending control input portion in a vertical direction along a longitudinal direction of the controller,
the air supply/water supply button and the suction button are arranged side by side in the vertical direction of the controller or in a horizontal direction perpendicular to the vertical direction.

17. The controller according to claim 16, wherein the bending control input portion is one of a touch pad, a lever, and a joystick.

18. The controller according to claim 17, wherein
the bending control input portion includes a touch pad, a vertical operation lever that bends the bending portion in the vertical direction, and a left-right operation lever that bends the bending portion in left-right direction, and
the vertical operation lever and the left-right operation lever are arranged side by side on both sides of the touch pad and in the left-right direction of the controller.

19. The controller according to claim 17, wherein
the controller includes a touch pad and a joystick as the bending control input portion, and
the touch pad and the joystick are arranged next to each other on a same surface of the controller.

20. The controller according to claim 17, wherein the controller includes a handle or a hanger through which a finger of a surgeon can be inserted into a surface facing a surface on which the bending control input portion is arranged.
